(19) United States
(12) Patent Application Publication
(10) Pub. No.: US 2024/0180826 A1
(43) Pub. Date: Jun. 6, 2024

(54) Opthalmic Compositions for Dry Eye Treatment and Methods of Manufacture of the Same

(71) Applicant: Marc Selner, North Hills, CA (US)

(72) Inventor: Marc Selner, North Hills, CA (US)

(21) Appl. No.: 18/530,529

(22) Filed: Dec. 6, 2023

Related U.S. Application Data
(60) Provisional application No. 63/475,701, filed on Dec. 6, 2022.

(51) Int. Cl. A61K 9/08; A61K 9/00; A61K 38/13; A61K 47/10; A61K 47/18; A61K 47/20; A61K 47/26; A61K 47/36

(52) U.S. Cl. CPC A61K 9/08; A61K 9/0048; A61K 38/13; A61K 47/10; A61K 47/183; A61K 47/20; A61K 47/26; A61K 47/36

(57) ABSTRACT

This invention presents novel ophthalmic formulations featuring cyclosporine to address eye irritation linked to traditional dry eye treatments. The formulation incorporates pure cyclosporine A and achieves water solubility without the need for lipids or emulsifiers, enhancing bioactivity. Preferred embodiments include moisturizing agents and osmolarity adjusters to improve efficacy. The invention provides two formulation methods, both excluding fats and emulsifiers for water solubility.

### TECHNICAL FIELD

The present disclosure relates to a water soluble solution prepared without emulsifiers or fats and methods to produce the same, and, more particularly, a treatment using that solution to be applied to the surface of the human eye for treatment of dry eye (keratoconjunctivitis sicca).

### CROSS REFERENCE TO RELATED APPLICATIONS

This application claims the benefit of U.S. Provisional Patent Application Ser. No. 63/475,701, entitled “Water soluble cyclosporine a dry eye and allergic eye,” filed on Dec. 6, 2022, the disclosure of which is hereby incorporated herein by reference thereto.

### BACKGROUND OF THE INVENTION

The eye produces tears that are spread across the eye while blinking. The unique components of tears combined with the blinking process create a tear film that is made up of an inner mucin layer, a middle aqueous layer, and an outer lipid layer. Mucin is a glycoprotein constituent of mucus and is a hydrophilic molecule. The inner mucin layer is produced by goblet cells in the eye. This inner mucin layer is right next to the conjunctiva and aids in spreading tears and stabilizing the tear film. In turn, this helps to prolong the tear break-up time. The middle aqueous layer is created by the lacrimal gland and is the largest portion of the tear film. It consists of a variety of different types and concentrations of mucins, proteins, peptides, enzymes, metabolites, and electrolytes. Most of the tear film components are dissolved in this layer. Finally, the outer lipid layer is manufactured by meibomian glands. This layer contains polar and non-polar lipids and prevents the evaporation of tears.

This tear film undergoes significant forces that can compromise the integrity of the film including: 1) evaporation, 2) spreading along the ophthalmic surface, which is driven by high shear blinks, 3) draining, which is aided by blink powered lacrimal pumping, and 4) low shear flow along the lid tear menisci. To maintain the tear film, the film is continually replenished with new tear film components upon each blink, which is triggered by tear breakup and corneal surface nerve excitation. This unique system creates a barrier between the environment and the surface of the eye and removes any irritants that may enter the eye. Further, tears have critical components derived from the blood plasma that are filtered to nourish the ophthalmic surface, reduce infection risk and promote healing of ophthalmic surface tissues. The tear film is the single most important optical surface. Disturbances that affect the quality and duration of that film on the cornea can dramatically alter quality of vision.

Keratoconjunctivitis sicca, or dry eye syndrome, is a severe event that can cause a reduction in the quantity or quality of tears. It is a common affliction that is caused by the failure of the eye to produce either an adequate amount or maintain a proper balance of tear components in the mucin, aqueous, or lipid layers. In either instance, the tear film that normally covers the eye, no longer covers the entire eye evenly and for a sufficient period (roughly eight seconds). Tear film instability causes tears to bead up leaving dry spots in the film and simultaneously failing to remove irritants. These dry spots and irritants cause many of the conditions associated with dry eye (e.g., burning, stinging, itching and tired eyes). The salinity of the tears themselves also increases and as small as a 10% salinity increase produces inflammation and damage to the eye, more particularly to the kerato—conjunctival layer of the eye. Damage can also occur to the tear film producers themselves—the goblet cells, lacrimal glands, and meibomian glands.

Dry eye symptoms can be exacerbated by activities that extend the time between eye blinks including prolonged computer use and reading. Even mild tear film degradation can reduce the tear break up time leading to excessive blinking, which may merely provide short term relief. Dry eye may occur for a variety of reasons including, for example, as a side effect of certain corneal surgeries, hormonal changes, autoimmune diseases, inflamed eyelid glands, allergic eye disease, and changes in environmental factors including air temperature and humidity.

Treatment for dry eye generally consists of artificial tear compositions which include immune modulators (e.g., cyclosporine) to reduce inflammation, moisturizers, osmolarity protectants, and polymers which act to mimic the mucin, aqueous, or lipid layers of the tear film to maintain the stability of the film and prevent rapid evaporation. High viscosity artificial tear compositions maintain a longer lasting tear film. However, these compositions cause viscous drag on the eyelids while blinking creating an uncomfortable “sticky” sensation, may be difficult to apply and creates a crust on the eyelids. These high viscosity compositions also result in blurred vision for several minutes or longer. Low viscosity compositions do not maintain a long-lasting tear film.

Current artificial tear compositions for treating dry eyes maintain a stable tear film for only a short period of time (about fifteen to twenty-five minutes or less), cause blurred vision (and in some cases can cause blindness), don't provide an evaporative shield or have a synthetic or oily feeling from added lipids or lipid-like substances that do not stabilize the aqueous layer, do not provide a protective coating over the conjunctiva of the lids and or sufficiently dissolve lipid inspissation within meibomian glands, do not provide a physiologically enhanced environment for epithelial cell healing, do not prevent, reduce or help dissolve protein, cholesterol, or dried mucous that may deposit on contact lens surfaces, the corneal epithelium, or the conjunctiva of the lid and irritate or otherwise degrade these cell membranes, they do not significantly promote tear secretion or provide prolonged exposure to and retention of existing tears, and they result in higher osmolarity and wetting angle making tear spread more difficult and uneven.

Cyclosporine (also referred to as pure cyclosporine A) is a 1-amino acid cyclic peptide immunosuppressant, and excessive prolonged use can cause side effects including kidney and liver complications, tumor growth, and infections. pure cyclosporine A is generally taken orally and reduces T-cell immunity in a patient, thus preventing organ rejection. It has been used as an effective treatment for dry eye when used in the appropriate quantity. This treatment has the potential to reverse eye damage, increase the number of goblet cells by 100 ppm, and enhance secretions from the lacrimal glands, as evidenced by the Schirmer's test. Cyclosporine offers several advantages, one of which is its suitability for long-term use. In contrast, cortisone must typically be discontinued after a few weeks to avoid potential complications, such as atrophy and hormonal effects, among others.

In the current artificial tear compositions, cyclosporine is dissolved in an oil or fat compound in an effort to replace meibomian function. For example, Restasis® uses 500 ppm of cyclosporine dissolved in castor oil with added emulsifiers; Cequa® uses 900 ppm of cyclosporine dissolved in castor oil with added emulsifiers; Miebo™ (recently sold to Klarity C®) consists of dissolved cyclosporine in a semi fluorinated alkane (perfluorohexyloctane) in an anhydrous formula, and Klarity C® uses 1000 ppm of cyclosporine dissolved in vinyl alcohol, castor oil, added emulsifiers, and chondroitin sulfate.

### SUMMARY OF THE INVENTION

The present invention provides a novel ophthalmic formulation containing cyclosporine as the active ingredient. The formulation addresses the issue of eye irritation caused by traditional dry eye treatments, particularly those containing lipids and emulsifiers. The proposed invention aims to provide a water-soluble formulation of cyclosporine, effectively eliminating the need for eye-irritating compounds, such as lipids and emulsifiers, while maintaining the drug's therapeutic efficacy.

More specifically, this formula incorporates pure cyclosporine A as the active ingredient, serving as an immune modulator to combat dry eye inflammation without fats or emulsifiers to dissolve the cyclosporine A. As a result, it achieves water solubility and seamlessly integrates with tears. This enhances its bioactivity and effectively reduces the signs and symptoms of dry eye. Preferred embodiments may include moisturizing agents such as hyaluronic acid, propylene glycol, and glycerin to hydrate the eye. To address hyperosmolarity (tears becoming too salty), preferred embodiments also incorporate osmolarity adjusters such as erythritol, L-carnitine, and betaine, to help restore the tear balance. In an alternative embodiment, the formulation utilizes commercially available non-modified cyclosporine in a capsule which also contains sodium lauryl sulfate to achieve aqueous solubility.

Provided are also two methods for formulation. For the first method, fat-soluble pure cyclosporine A is dissolved in a small amount of ethanol, then PHMB (Polyhexamethylene Biguanide) and water are introduced before the solution undergoes a vacuum process to remove air. In an alternative embodiment, the sodium lauryl sulfate present in a non-modified cyclosporine capsule aids in solubilizing pure cyclosporine A. Notably, this formula avoids the use of fats and, consequently, emulsifiers, making it water-soluble and biocompatible with tears.

### BRIEF DESCRIPTION OF THE DRAWINGS

The inventive ophthalmic compositions for the treatment of dry eye and methods of manufacture of the compositions will become apparent from the following description taken in conjunction with the drawings, in which:

FIG. 1 is a cross section of the tear film of a human eye.

### DETAILED DESCRIPTION OF THE PREFERRED EMBODIMENTS

The inventive composition comprises a novel water-soluble ophthalmic formula for treating dry eye with cyclosporine as the primary active ingredient. The innovation encompasses not only the formulation itself but also multiple methods for its preparation.

### I. STRUCTURE OF THE EYE

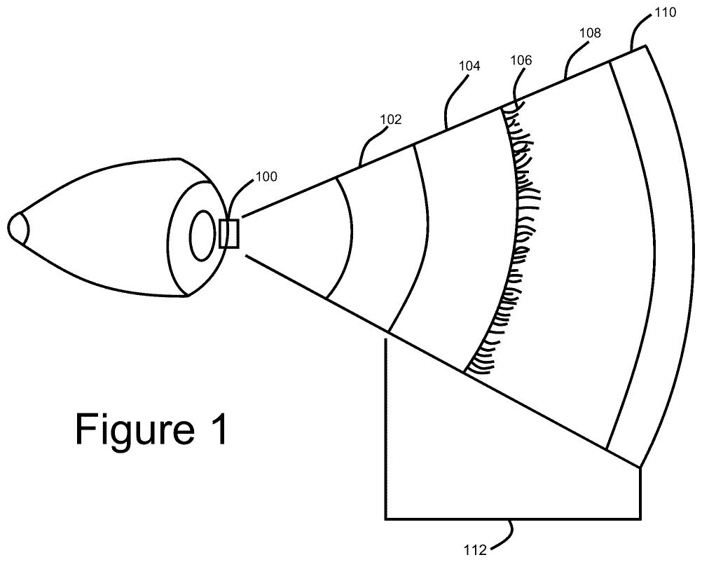

FIG. 1 is a cross section of the surface of a human eye 100 which shows the corneal epithelium 102, the tear film itself 112 and the three layers of the tear film including the mucin layer 104, the aqueous layer 108 and the lipid layer 110. There are a plethora of microvilli 106 present in the aqueous layer. As mentioned above, dry eye is a superficial disease occurring on the surface of the eye. As such, it is pertinent that any treatment for dry eye does not penetrate the eye beyond the tear film 112. The present invention has the ability to mix with the tear film 112 promoting relief for the signs and symptoms of dry eye. Current products on the market (e.g., Restasis®) do not mix with the tear film because they are lipid-soluble formulations, or anhydrous formulations, and can even interfere with the tear film to such an extent that blurry vision and temporary blindness can occur.

Notably, this formula avoids the use of fats and, consequently, emulsifiers, making it water-soluble and biocompatible with tears. Tears consist of three layers: mucin produced by goblet cells, saltwater from the lacrimal glands (0.6 mg per milliliter), and any increase beyond 10% in salt concentration can lead to eye damage.

As noted above, cyclosporine is an immunosuppressive drug commonly employed in various ophthalmic solutions designed to address dry eye conditions. Nevertheless, these products, such as Restasis®, Cequa®, and Miebo™, employ manufacturing methods that result in remarkably low bioavailability of cyclosporine. This limitation is underscored by FDA trials that have shown Restasis® to be ineffective in alleviating the signs and symptoms of dry eye. The reduced efficacy is attributed to the diminished bioavailability of cyclosporine when it is dissolved in lipid emulsifiers. As a result, these formulations require considerably higher doses of cyclosporine, which can increase the risk of severe side effects. These potential side effects may include seizures, severe infections, abrupt kidney injury, liver damage, nerve complications, allergic reactions, and more. Thus, both Restasis® and Cequa® exhibit limitations in effectively alleviating dry eye symptoms. Despite their widespread use, these medications primarily enhance tear production 15% after a six-month duration. The critical concern is that dry eye signs and symptoms, including redness, inflammation, light sensitivity, and a burning sensation, persist, casting doubt on treatment effectiveness and leaving room for improvement.

Emulsifiers and lipids have conventionally been employed in dry eye treatments due to their ability to aid in dissolving hydrophobic compounds like cyclosporine. These substances play a vital role in enhancing the bioavailability and effectiveness of cyclosporine for treating dry eye syndrome. However, there is a drawback—they can potentially irritate the eyes of certain individuals. Notably, lipids can form a sticky film on the ocular surface, leading to blurred vision and discomfort. Despite these issues, recent formulation Miebo™ uses an anhydrous formula, failing to address these issues.

In contrast, the present invention, because of its water solubility, easily combines with tears, enhancing its bioactivity by removing the fat component. The fat-free cyclosporine remains highly active. It is effective in alleviating the signs and symptoms of dry eye for an extended period. Moreover, the low dose of cyclosporine allows for long-term use without adverse effects. Additionally, the use of PHMB as a preservative ensures that the shelf stability extends over several years, provided there is no contamination when the dropper comes into contact with the eye. There is further provided a method of manufacture where cyclosporine is dissolved in ethanol, by adding PHMB and water, and then sealing the cyclosporine-ethanol-PHMB mixture into a sealed vacuum chamber to further promote solubility. This process shifts the polarity of cyclosporine allowing it to remain dissolved in water even after the remaining ethanol in the mixture is distilled out. Moisturizing agents (e.g., glycerin, propylene glycol, and hyaluronic acid) and osmolarity protectants (e.g., erythritol, betaine, and l-carnitine) are added to the final solution in order to further aid in treating the signs and symptoms of dry eye.

### II. COMPOSITIONS AND METHODS FOR MAKING THE SAME

There are three kinds of commercially available cyclosporine: (1) pure cyclosporine A (e.g., Sandimmune®), and (2) modified cyclosporine (e.g., Neoral®) and non-modified cyclosporine A (typically in a capsule form for oral administration). Pure cyclosporine is traditionally used in ophthalmic solutions for the treatment of dry eye. Both non-modified cyclosporine and pure cyclosporine A contain the same active ingredient of cyclosporine but are absorbed differently in the body when taken orally. When administered to the eye's surface, both non-modified and pure cyclosporine A exhibit an equal level of effectiveness in the treatment of dry eye.

The invention provides for compositions that contain pure cyclosporine A as an active ingredient solubilized in water. The characteristics of a water-soluble pure cyclosporine A composition differ from prior art compositions where pure cyclosporine A is solubilized in lipids and maintained in solution via emulsifiers.

In a first preferred embodiment of the composition, the ophthalmic solution comprises 0.005%-0.1% pure cyclosporine A by weight. To make this solution 5-1000 mg, preferably 50-100 mg pure cyclosporine A solubilized in ethanol and then mixed into 1000 ml water, using an amount as low as 5 ppm of pure cyclosporine A as compared to other compositions on the market which use upwards of 500-800 ppm of pure cyclosporine A all of which have less bioactivity than the present disclosure because they are lipid based. The amount of ethanol needed to solubilize pure cyclosporine A varies but typically around 4 ml of ethanol is needed to solubilize 100 mg of pure cyclosporine A. Therapeutic levels of pure cyclosporine A range from 0.005% to 0.1% by weight. The greater the concentration of pure cyclosporine A in the solution, the higher the likelihood of a patient experiencing adverse side effects. Furthermore, a pure cyclosporine A concentration exceeding 0.1% by weight typically renders pure cyclosporine A insoluble in an aqueous solution, while concentrations lower than 0.005% by weight result in negligible therapeutic efficacy.

A remarkable aspect of the first preferred method is its capacity to alter the polarity of cyclosporine, transforming it from lipid-soluble to water-soluble. This transformative process occurs when cyclosporine is combined with a suitable organic alcohol, such as ethanol. Notably, this composition stands out by its absence of fats and emulsifiers, a departure from conventional ophthalmic solutions. An unexpected advantage of this innovation is its ability to achieve superior bioactivity while utilizing a reduced quantity of cyclosporine compared to prior art treatments such as Restasis® and Cequa®, making it exceptionally effective in addressing the signs and symptoms of dry eye and safer for the patient.

In preferred embodiments, the concentration ranges of key are as follows. Pure cyclosporine A is included at a concentration range of 0.005% to 0.1%. Ethanol is incorporated in the formulation at a concentration of 0.05% to 0.4%. The antimicrobial agent PHMB is present within the range of 0.02% to 0.06%. Glycerin, Propylene Glycol, Hyaluronic Acid, Betaine, L-Carnitine, and Erythritol contribute to the formulation at concentrations ranging from 0.1% to 1.0%, 0.1% to 1.0%, 0.1% to 1.0%, 0.02% to 0.25%, 0.05% to 0.5%, and 0.05% to 0.5%, respectively. The majority of the formulation consists of water, making up 95% to 98% of the total composition. While this is illustrative of ingredient concentrations, it is understood that a person skilled in the art would determine precise over each component to achieve the desired pharmaceutical characteristics.

In a second preferred embodiment, pure cyclosporine A is used both in its pure form and the non-modified form. Non-modified cyclosporine capsules, are available in 25 mg and 100 mg strengths. Each capsule typically contains the following inactive ingredients: methanol, sodium lauryl sulfate and talc. Sodium lauryl sulfate is an anionic detergent and surfactant which lowers the surface tension of the solution, thus promoting the solubility of cyclosporine in an aqueous solution. This method utilizes mixing cyclosporine with sodium lauryl sulfate lowering the surface tension of the solution thus transforming cyclosporine from lipid-soluble to water-soluble. The sodium lauryl sulfate is an anionic detergent and surfactant which lowers the surface tension of the solution allowing cyclosporine to dissolve readily in water.

A non-modified cyclosporine PHMB solution is made using the contents of a standardly available capsule of non-modified cyclosporine (comprising pure cyclosporine A, methanol, sodium lauryl sulfate and talc) is solubilized in PHMB and water, aided by the already contained therein 0.003% by weight sodium lauryl sulfate which lowers the surface tension of the solution. As sodium lauryl sulfate can be irritating to some patients, for end use, the sodium lauryl sulfate can be further diluted by a factor of 1000 to 0.00003% by dissolving pure cyclosporine A into about 1% of the non-modified cyclosporine PHMB solution. For example, take 10 ml of the 0.003% non-modified cyclosporine PHMB solution and add in 100 mg of pure cyclosporine A to solubilize it and resulting in a batch with only 0.00003% sodium lauryl sulfate. In an alternative embodiment sodium lauryl sulfate is diluted to between 0.03 to 0.0003%. The 0.00003% by weight of sodium lauryl sulfate remains in solution, thus maintaining the lower the surface tension of the solution which continuously promotes the solubility of pure cyclosporine A. Both the pure and non-modified cyclosporine molecules continue to stay in a dissolved state within the diluted solution.

In an alternative embodiment, 0.005% to 0.1% by weight of pure cyclosporine A is dissolved in 10 ml of dissolved non-modified cyclosporine, PHMB, and enough distilled water so that the sodium lauryl sulfate is in a concentration preferably around 0.00003%. In preferred embodiments, propylene glycol and glycerin may be added in an amount 0.1% to 1.0% by weight.

In connection with the formulations described above, the composition may also contain one or more water-soluble excipients which aid in relieving the signs and symptoms of dry eye. These excipients preferably include 0.1% to 1.0% moisturizing agents by weight. Preferred agents include glycerin which can be used as both a moisturizer and an osmolarity protectant, propylene glycol which can be used as a moisturizer, and hyaluronic acid which can be used both a moisturizer and an osmolarity protectant) to aid in increasing moisture into the eye by strengthening the tear film. The preferred range for these moisturizing agents is about 0.1% to 1.0% by weight, due to their therapeutic effectiveness. Insufficient amounts will likely have negligible benefits, while concentrations exceeding 1.0% by weight can lead to eye irritation, a burning sensation, and blurred vision. Furthermore, as the concentration of these moisturizing agents increases, the solution's viscosity rises, hindering its ability to blend effectively with the tear film.

These excipients may also include additional osmolarity protectants (e.g., betaine, l-carnitine, and erythritol) to prevent a fluctuation in the salinity of tears. Betaine is a surfactant, antimicrobial, biocide, and osmolarity protectant. As such, and because it is used in combination with another biocide, it should be used sparingly, preferably 0.02%-0.25% by weight so as to optimize its therapeutic effects with as little irritation as possible. The remaining osmolarity protectants (e. g., l-carnitine and erythritol) are present ranging from 0.05%-0.5% by weight to optimize their therapeutic effect and prevent irritation, burning, blurriness, or an over thickening of the tear film.

PHMB which functions as an antimicrobial about 0.02% to 0.06% (PHMB) by weight which has a dual function to both prevent infection which can occur from complications of dry eye and as a preservative for the composition which keeps it shelf stable for at least two years.

Ethanol, preferably pure 200 proof, is used as a solvent for the pure cyclosporine A. 100 mg of pure cyclosporine A is dissolved in 3-4 ml of ethanol. Other organic alcohols that are ophthalmically safe and will dissolve pure cyclosporine A include up to 0.05% isopropyl alcohol by weight, up to 0.05% benzyl alcohol by weight, and 0.05% DMSO by weight. It is understood that any unbound alcohol will evaporate.

Other ingredients may be included in the composition and can include alternative moisturizing agents (e.g., 0.1% to 0.25% by weight dextrose, 1% to 95% by weight purified water, 0.1% to 0.25% by weight ethyl cellulose, 0.1% to 0.25% by weight methyl cellulose, 0.1% to 0.25% by weight carboxymethylcellulose, 0.1% to 0.25% by weight polysorbate, 0.1% to 0.25% by weight guar, 0.1% to 0.25% by weight polyethylene glycol), alternative osmolarity protectants (e.g., glycerin in an amount of 0.1% to 1.0%, and hyaluronic acid in an amount of 0. 1% to 0.25%), an alternative aqueous biocide antimicrobial (e.g., providone-iodine in an amount of 0.1% to 1.0% by weight, isopropyl alcohol in an amount of 0.001% to 0.005% by weight, vinyl alcohol in an amount of 0.001% to 0.005% by weight, betaine in an amount of 0.02% to 0.25% by weight (preferably 0.05% due to potential irritation in combination with any other additive), benzalkonium chloride in an amount of 0.001% to 0.002% by weight, boric acid in an amount of 0.001% to 0.002% by weight, and polyquat in an amount of 0.001% to 0.002% by weight). These ranges are preferred but it is understood that these ranges can be modified depending on the final combination of the ingredients and such specifics would be determined by formulators skilled in the art. Once all the other ingredients are in, enough distilled water is added to make a 1 liter batch.

PHMB is the preferred biocide as it is a surfactant and will not penetrate the ophthalmic surface as dry eye is a surface level disease. The composition can further be tailored to include anti-allergens (e.g., Benadryl®) to simultaneously treat dry eye associated with allergies. The remaining weight of any embodiment of the present invention consists of water. All ingredients in the formula are FDA approved.

### III. ILLUSTRATIVE EXAMPLES

### Example 1

1. Place into a mortar and pestle 100 mg of pure cyclosporine A.
2. Add approximately 4 ml of 200-proof pure ethyl alcohol.
3. Stir to dissolve the pure cyclosporine A to make a solution.
4. In a separate container add four drops of 20% PHMB to one liter of demineralized water to make an aqueous biocide solution.
5. Add around 50 ml of the aqueous biocide solution to the solution and stir to make an aqueous mixture.
6. Place the mortar with the aqueous mixture in a small hand vacuum at (−)15-75 Pa for 15 to 60 minutes.
7. Remove the mortar with the aqueous mixture from the vacuum and stir.
8. Heat the aqueous mixture to the boiling point of ethanol, 90° F. Add the remaining 950 ml of the PHMB solution to bring the total volume of the aqueous mixture up to 1 liter to make an ophthalmic solution.

Optionally the following excipients may be added, including moisturizers and osmolarity agents, which are all water-soluble:
a. Add 0.5% of propylene glycol by weight.
b. Add 0.5% of glycerin by weight.
c. Add 0.25% of hyaluronic acid by weight.
d. Add 0.025% of erythritol by weight.
e. Add 0.25% of L-carnitine by weight.
f. Add 0.05% of betaine by weight.
2. Stir the solution thoroughly to complete the manufacturing process.

The pure cyclosporine A remains in a dissolved state at an ionic level after rehydration. A small quantity of ethanol remains attached to cyclosporine, having been distilled off at its boiling point within the range of 85-90° F. This residual ethanol serves to maintain the formulation's polarity, rendering it primarily water-soluble. This property enables effective mixing with tears and enhances its bioactivity. Additionally, when the initial aqueous solution is placed in the small hand vacuum, any remaining air in the solution is removed, further promoting solubility. The amount of pure cyclosporine A is so minimal that it cannot be detected in blood levels. There is no free ethanol in the solution; any residual ethanol that isn't distilled off is attached to pure cyclosporine A. The removal of air from the water during the vacuum step increases the solubility of the pure cyclosporine A. Additionally, three moisturizers—propylene glycol, glycerin, and hyaluronic acid—are preferably included in the formulation to enhance its ophthalmic properties. Similarly, to enhance the formulation's ophthalmic properties, three osmolarity protectants—betaine, l-carnitine, and erythritol—are preferably included as well.

### Example 2

Below is an illustrative example of a second manufacturing method of the present invention using readily available and FDA-approved non-modified pure cyclosporine A. A base solution is prepared with non-modified pure cyclosporine A, and pure cyclosporine A can be subsequently added to it. This method utilizes encapsulated non-modified pure cyclosporine A and sodium lauryl sulfate, both conveniently obtainable in FDA-approved forms.

### Base Solution:

1. Begin with a capsule of 100 mg of non-modified pure cyclosporine A, containing both pure cyclosporine A and sodium lauryl sulfate. Place the contents of the capsule into a mortar and pestle.
2. In a separate container add about four drops of 20% PHMB to one liter of demineralized water.
3. Stir to mix the PHMB with the demineralized water to make an aqueous biocide solution.
4. Add 50 ml of the aqueous biocide solution to the contents of the capsule into the mortar.
5. Stir to dissolve the non-modified pure cyclosporine A to make an ophthalmic solution.
6. Add the moisturizing agents to make a base solution:
   a. Add 0.5% of propylene glycol.
   b. Add 0.5% drops of glycerin.
   c. Add 0.5% of hyaluronic acid.

### Final Solution:

7. Place 100 mg of pure cyclosporine A into a mortar.
8. Add 10 ml of the base solution into the mortar.
9. Stir to dissolve the pure cyclosporine A.
10. In a separate container add about four drops of 20% PHMB to one liter of demineralized water to make an aqueous biocide solution.
11. Add the aqueous biocide solution into the mortar, reducing the concentration of sodium lauryl sulfate to avoid eye irritation to make an ophthalmic solution.
12. Add the excipients, including moisturizing agents and osmolarity protectants, to the ophthalmic solution:
   a. Add 0.5% of propylene glycol.
   b. Add 0.5% drops of glycerin.
   c. Add 0.5% of hyaluronic acid.
   d. Add 0.25% of l-carnitine.
   e. Add 0.05% of betaine.
   f. Add 0.25% of erythritol.

The use of both non-modified and pure cyclosporine A in this method is attributed to their ability to remain water-soluble and stay in solution upon rehydration. This is primarily facilitated by the presence of sodium lauryl sulfate, an anionic detergent and surfactant, which reduces the surface tension of the solution, thus promoting solubility. However, it's important to note that sodium lauryl sulfate can be irritating to the eye. In the preferred alternative formulation, it is diluted with a liter of water to lower its concentration, thereby preventing potential irritation. Alternatively, those skilled in the art can formulate this solution by directly adding sodium lauryl sulfate to pure cyclosporine A for dissolution in water, eliminating the need for a specific initial batch. In an alternative approach, sodium lauryl sulfate is used instead of ethanol in the formulation.

### IV. IMPROVED EFFICACY

Cyclosporine serves as an anti-inflammatory agent, distinct from cortisone, and can be used for extended periods due to its immune-modulating properties. It readily mixes with tears, comprising two layers: the lacrimal tears containing salty water and proteins, and the mucin from goblet cells forming the first two layers. The third layer, myobin, is fat-soluble and acts as a barrier to prevent tear evaporation.

The present composition provides a precise and targeted delivery to the affected area, namely, the surface of the eye. Everything relevant in treating dry eye is concentrated at the surface. The epithelial cells, meibomian glands, and other critical components are all located at this surface layer. The epithelium of the eye, consisting of around 5 to 6 clear layers, is particularly delicate. Hyperosmolarity can cause thinning, lifting, and even pitting, leading to visible erosion into the eye. The use of dyes can make these effects apparent. In contrast to prior art formulations that contain mixed fats, which can obstruct the active ingredient's access to the intended target, this formulation eliminates emulsifiers. Consequently, it achieves greater effectiveness with less cyclosporine, ensuring that the treatment reaches the precise area where it is needed most.

Through the development of an aqueous solution containing cyclosporine, devoid of eye-irritating compounds like lipids and emulsifiers, this innovation represents a notable advancement in dry eye management. Its distinctive composition and advantages underscore its potential to enhance the quality of life for numerous patients. This formulation offers a safer and more effective alternative for individuals suffering from dry eye. By eliminating components known to cause eye irritation, it significantly enhances patient compliance and comfort. The absence of lipids and emulsifiers ensures efficient drug delivery to the ophthalmic surface, resulting in improved therapeutic outcomes.

Furthermore, its water solubility facilitates seamless integration with tears, enhancing its therapeutic potential by eliminating fat-based components. This fat-free cyclosporin remains highly effective, providing relief for the signs and symptoms of dry eye over an extended period. The low cyclosporine dose enables long-term use without adverse effects. The use of PHMB as a preservative ensures extended shelf stability for several years, contingent on preventing contamination when the dropper makes contact with the eye.

While illustrative embodiments of the invention have been described, it is noted that various modifications will be apparent to those of ordinary skill in the art in view of the above description and drawings. Such modifications are within the scope of the invention which is limited and defined only by the following claims.

What is claimed:

1. An aqueous topical ophthalmic solution for treating an eye of a human having keratoconjunctivitis sicca, wherein the solution comprises:
a) a first solute comprising cyclosporine or acceptable salt thereof,
b) said solute dissolved into a solvent comprising
i) an ophthalmically safe organic alcohol
ii) water
wherein the aqueous ophthalmic solution is therapeutically effective in treating keratoconjunctivitis sicca.

2. The solution of claim 1, wherein the ophthalmically safe organic alcohol is selected from the group consisting of ethanol, isopropyl alcohol, and benzyl alcohol.

3. The solution of claim 1, further comprising at least one of the following a moisturizing agent, an osmolarity protectant, or an aqueous biocide.

4. The solution of claim 3, wherein the moisturizing agent is selected from the group consisting of propylene glycol, hyaluronic acid, dextrose, purified water, ethyl, methyl cellulose, carboxymethylcellulose, polysorbate, guar, glycerin, and polyethylene glycol; wherein the osmolarity protectant is selected from the group consisting of betaine, l-carnitine, erythritol, glycerin, and hyaluronic acid; and wherein the aqueous biocide is selected from the group consisting of PHMB, providone-iodine, vinyl alcohol, betaine, benzalkonium chloride, borate, boric acid, and polyquat.

5. An aqueous topical ophthalmic solution for treating an eye of a human having keratoconjunctivitis, wherein the topical ophthalmic comprises:
a) cyclosporine A;
b) sodium lauryl sulfate; and
c) water
wherein the ophthalmic solution is therapeutically effective in treating keratoconjunctivitis sicca.

6. The solution of claim 5, further comprising at least one of the following a moisturizing agent, an osmolarity protectant, or an aqueous biocide.

7. A method to manufacture an aqueous topical ophthalmic solution for treating an eye of a human having keratoconjunctivitis sicca, wherein said ophthalmic solution is formulated by the steps of:
a) dissolving pure cyclosporine A into an ophthalmically safe alcohol to make a first solution;
b) adding water to make a second solution;
c) applying vacuum to the second solution until any remaining pure cyclosporine A is visibly dissolved; and
d) heating the solution to the boiling point of the alcohol to evaporate any free alcohol wherein the ophthalmic solution is therapeutically effective in treating keratoconjunctivitis sicca.

8. The topical ophthalmic solution manufactured by the process of claim 7.

9. The method of claim 7, wherein the biocompatible alcohol is selected from the group consisting of ethanol, isopropyl alcohol, and benzyl alcohol.

10. The method of claim 7, further comprising the additional step of mixing in a biocide solution to the second solution and then putting under said vacuum.

11. The method of claim 10, wherein the aqueous biocide solution comprises:
a) water; and
b) an aqueous biocide selected from the group consisting of PHMB, providone-iodine, vinyl alcohol, betaine, benzalkonium chloride, borate, boric acid, and polyquat.

12. The method of claim 7, further comprising the additional step adding one more of the following additives: a moisturizing agent, an osmolarity protectant, or an aqueous biocide.

13. The method of claim 12, wherein said moisturizing agent is selected from the group consisting of propylene glycol, hyaluronic acid, dextrose, purified water, ethyl, methyl cellulose, carboxymethylcellulose, polysorbate, guar, glycerin, and polyethylene glycol; wherein said osmolarity protectant is selected from the group consisting of betaine, l-carnitine, erythritol, glycerin, and hyaluronic acid.

14. A method to manufacture an aqueous topical ophthalmic solution for treating an eye of a human having keratoconjunctivitis sicca, wherein said ophthalmic solution is formulated by the steps of
a) mixing cyclosporine A, methanol, sodium lauryl sulfate and talc into a powder;
b) mixing PHMB and demineralized water to make aqueous biocide solution;
c) adding the aqueous biocide solution to the powder; and
d) stirring to dissolve the powder into the aqueous biocide solution to make an ophthalmic solution,
wherein the concentration of pure cyclosporine A is 100 ppm and wherein the ophthalmic solution is therapeutically effective in treating keratoconjunctivitis sicca.

15. The topical ophthalmic solution manufactured by the process of claim 14.

16. The method of claim 14, further comprising the additional step of dissolving pure cyclosporine A into the aqueous topical ophthalmic solution, then adding an aqueous biocide solution to dilute the maximum concentration of a and wherein the concentration of pure cyclosporine A is 100 ppm and is therapeutically effective in treating keratoconjunctivitis sicca.

17. The method of claim 14, wherein the biocide solution comprises
a) water; and
b) a biocide selected from the group consisting of PHMB, providone-iodine, vinyl alcohol, betaine, benzalkonium chloride, borate, boric acid, and polyquat.

18. The method of claim 14, further comprising the additional step adding one more of the following additives: a moisturizing agent, an osmolarity protectant, or an aqueous biocide.

19. The method of claim 18 wherein said moisturizing agent is selected from the group consisting of propylene glycol, hyaluronic acid, dextrose, purified water, ethyl, methyl cellulose, carboxymethylcellulose, polysorbate, guar, glycerin, and polyethylene glycol, wherein said osmolarity protectant is selected from the group consisting of betaine, l-carnitine, erythritol, glycerin, and hyaluronic acid.
